(19) United States
(12) Patent Application Publication
(10) Pub. No.: US 2025/0281609 A1
(43) Pub. Date: Sep. 11, 2025

(54) COMPOSITION FOR TREATING OR PREVENTING CANCER

(71) Applicant: ORGANOIDSCIENCES LTD., Seongnam-si, Gyeonggi-do (KR)

(72) Inventors: Yoori SHIN, Seongnam-si (KR); Dong Hyeon KIM, Seongnam-si (KR); Bo Eun LEE, Seongnam-si (KR); Sarang KIM, Seongnam-si (KR); Jihyo KIM, Seongnam-si (KR)

(73) Assignee: ORGANOIDSCIENCES LTD., Seongnam-si, Gyeonggi-do (KR)

(21) Appl. No.: 18/703,009

(22) PCT Filed: Oct. 19, 2022
(86) PCT No.: PCT/KR2022/015984

§ 371 Date: Apr. 9, 2025

(30) Foreign Application Priority Data
Oct. 19, 2021 (KR) 10-2021-0139308

(51) Int. Cl. A61K 39/42; A61K 31/395; A61K 31/4025; A61K 31/4196; A61K 31/496; A61K 31/519; A61K 31/52; A61K 31/5377; A61P 35/00; G01N 33/50

(52) U.S. Cl. CPC A61K 39/42; A61K 31/395; A61K 31/4025; A61K 31/4196; A61K 31/496; A61K 31/519; A61K 31/52; A61K 31/5377; A61P 35/00; G01N 33/5011

(57) ABSTRACT

The present invention relates to a composition comprising an inhibitor of herpesvirus entry mediator (HVEM) or its ligand and a heat shock protein (HSP) inhibitor for treating or preventing cancer. The use of an inhibitor of HVEM or its ligand and an HSP inhibitor in combination exhibits a synergistic effect on anticancer activity, compared to the use thereof singly, and shows an effect within as fast as 72 hours after administration. Therefore, the use of an inhibitor of HVEM or its ligand and an HSP inhibitor in combination can be advantageously utilized for preventing or treating cancer.

### TECHNICAL FIELD

The present invention relates to a composition for treating or preventing cancer, comprising an inhibitor of herpesvirus entry mediator (HVEM) or its ligand and a heat shock protein (HSP) inhibitor.

### BACKGROUND ART

Despite intensive research on cancer over the past several years, cancer remains a leading cause of death worldwide. Numerous cancer therapies have been developed, but they are not effective for all cancer types or for all patients. Most of the currently used methods for treating cancer are relatively non-selective. These include removing diseased tissue by surgery, reducing the size of solid tumors through radiation therapy, or rapidly inducing cancer cell death via chemotherapy. In particular, chemotherapy may lead to the development of drug resistance and, in some cases, cause severe side effects that limit the administrable dose, thereby precluding the use of potentially effective drugs. Accordingly, there is an urgent need to develop more effective and target-specific methods for cancer treatment.

Therefore, methods involving combination therapy with anticancer agents for the prevention or treatment of cancer have been developed. For example, a study analyzing the anticancer effect of rituximab (an anti-CD20 antibody) and venetoclax (a BCL2 protein inhibitor) has been reported (John F Seymour et al., N Engl J Med. 2018 Mar. 22, 378 (12): 1107-1120). In this study, the ‘venetoclax-rituximab’ combination therapy did not exhibit toxicity and showed superior anticancer effects compared to venetoclax monotherapy, with a 2-year rate of progression-free survival reaching 81.5%. However, it has been reported that, when bendamustine, which induces cancer cell death by binding to the DNA of cancer cells, is administered in combination with rituximab, the 2-year rate of progression-free survival was only approximately 27.8%.

This suggests that the combination of anticancer agents does not always result in synergistic effects. Thus, there is a strong need in the field to discover and propose optimal combinations of anticancer agents that can exert synergistic effects in cancer treatment.

### Technical Problem

The inventors conducted research to identify combinations of anticancer agents that could produce synergistic effects in anticancer activity. As a result, it was experimentally demonstrated that the combination of an inhibitor of HVEM or its ligand, and an HSP inhibitor, exhibited significantly increased anticancer activity compared to the use of each alone. In particular, it was confirmed that tumor growth can be suppressed at a very rapid rate within 72 hours, thus completing the present invention.

### Technical Solution

One aspect of the present invention provides a pharmaceutical composition for the prevention or treatment of cancer, comprising: (a) an inhibitor of herpesvirus entry mediator (HVEM) or its ligand; and (b) an inhibitor of heat shock protein (HSP).

The term “herpesvirus entry mediator (HVEM)” refers to a cell surface receptor protein belonging to the TNF receptor superfamily, encoded by the TNFRSF14 gene. It is also referred to as “Tumor Necrosis Factor Receptor Superfamily Member 14 (TNFRSF14)”. Specifically, the gene encoding HVEM may be or comprise a nucleic acid sequence encoding the amino acid sequence of human-derived HVEM, or may be or comprise the nucleic acid sequence of NCBI Reference Sequence: NM_001297605 or NM_003820 disclosed in NCBI. The nucleic acid sequence of HVEM may be or comprise a sequence having at least 80%, 85%, 90%, or 95% sequence identity with the nucleic acid sequences of NCBI Reference Sequence: NM_001297605 or NM_003820 disclosed in NCBI. However, the present invention is not limited thereto, and any nucleic acid sequence capable of producing an amino acid with the characteristics or functions of HVEM is encompassed. In addition, the amino acid sequence of HVEM may be or comprise the sequence of NCBI Reference Sequence: NP_001284534 or NP_003811 disclosed in NCBI. The amino acid sequence of HVEM may be or comprise a sequence having at least 80%, 85%, 90%, or 95% sequence identity with the sequences of NCBI Reference Sequence: NP_001284534 or NP_003811. However, the present invention is not limited thereto, and any amino acid sequence exhibiting the characteristics or functions of HVEM is encompassed.

Specifically, the HVEM inhibitor may bind directly to HVEM, bind directly to a ligand of HVEM, or inhibit the binding of HVEM to its ligand. Through such mechanisms, the activity of HVEM can be suppressed or inhibited, thereby leading to inhibition of tumor growth or induction of tumor cell death.

The term “ligand” refers to a substance that binds to a biomolecule such as a receptor protein to form a complex in order to perform a biological function, and include substrates, inhibitors, activators, signaling lipids, and neurotransmitters. Preferably, the ligand of HVEM may be one or more selected from the group consisting of B- and T-lymphocyte attenuator (BTLA) and LIGHT. The term “B- and T-lymphocyte attenuator (BTLA)” refers to a protein encoded by the BTLA gene, also known as “CD272”, and is known to be a ligand of HVEM. Specifically, the gene encoding BTLA may be or comprise a nucleic acid sequence encoding the amino acid sequence of human-derived BTLA, or may be or comprise the nucleic acid sequence of NCBI Reference Sequence: NM_001085357 or NM_181780 disclosed in NCBI. The nucleic acid sequence of BTLA may be or comprise a sequence having at least 80%, 85%, 90%, or 95% sequence identity with the nucleic acid sequence of NCBI Reference Sequence: NM_001085357 or NM_181780 disclosed in NCBI. However, the present invention is not limited thereto, and any nucleic acid sequence capable of producing an amino acid with the characteristics or functions of BTLA is encompassed. In addition, the amino acid sequence of BTLA may be or comprise the sequence of NCBI Reference Sequence: NP_001078826 or NP_861445 disclosed in NCBI. The amino acid sequence may be or comprise a sequence having at least 80%, 85%, 90%, or 95% sequence identity with NP_001078826 or NP_861445 disclosed in NCBI. However, the present invention is not limited thereto, and any amino acid sequence exhibiting the characteristics or functions of BTLA is encompassed.

The term “LIGHT” refers to a protein belonging to the tumor necrosis factor superfamily, also known as “Tumor Necrosis Factor Superfamily Member 14 (TNFSF14),” and is known to be a ligand of HVEM. Specifically, the gene encoding LIGHT may be or comprise a nucleic acid sequence encoding the amino acid sequence of human-derived LIGHT, or may be or comprise the nucleic acid sequence of NCBI Reference Sequence: NM_003807, NM_172014 or NM_001376887 disclosed in NCBI. The nucleic acid sequence of LIGHT may be or comprise a sequence having at least 80%, 85%, 90%, or 95% sequence identity with the nucleic acid sequence of NCBI Reference Sequence: NM_003807, NM_172014 or NM_001376887 disclosed in NCBI. However, the present invention is not limited thereto, and any nucleic acid sequence capable of producing an amino acid with the characteristics or functions of LIGHT is encompassed. In addition, the amino acid sequence of LIGHT may be or comprise the sequence of NCBI Reference Sequence: NP_003798, NP_742011 or NP_001363816 disclosed in NCBI. The amino acid sequence of LIGHT may be or comprise a sequence having at least 80%, 85%, 90%, or 95% sequence identity with the sequence of NCBI Reference Sequence: NP_003798, NP_742011 or NP_001363816. However, the present invention is not limited thereto, and any amino acid sequence exhibiting the characteristics or functions of LIGHT is encompassed.

The term “heat shock protein (HSP)” refers to a family of proteins that function to stabilize and assist in the proper folding of proteins damaged due to cellular stress. HSPs may be classified depending on molecular weight, such as HSP40, HSP60, HSP70, and HSP90. In an attempt to develop cancer therapies targeting HSPs, an HSP90 inhibitor known as Luminespib (AUY922) underwent clinical trials in patients with metastatic pancreatic adenocarcinoma; however, the trial was discontinued before the completion of Phase 2. As part of efforts to explore the utilization of drugs that have not been successfully developed as therapeutics, the present invention aimed to identify drug combinations that could exhibit synergistic anticancer activity when used in combination with HSP inhibitors.

Specifically, the HSP inhibitor may bind directly to HSP, bind directly to a ligand of HSP, or inhibit the binding of HSP to its ligand. Through such mechanisms, the activity of HSP can be suppressed or inhibited, thereby leading to inhibition of tumor growth or induction of tumor cell death. Preferably, the HSP may be one or more selected from the group consisting of HSP40, HSP60, HSP70 and HSP90.

The “HSP40” is also referred to as ‘chaperone DnaJ’, and the gene encoding HSP40 may be or comprise a nucleic acid sequence encoding the amino acid sequence of human-derived HSP40, or may be or comprise a nucleic acid sequence of NCBI Reference Sequence: NM_001313964.2, NM_001300914.2 or NM_006145.3 disclosed in NCBI. The nucleic acid sequence of HSP40 may be or comprise a sequence having at least 80%, 85%, 90%, or 95% sequence identity with the nucleic acid sequence of NCBI Reference Sequence: NM_001313964.2, NM_001300914.2 or NM_006145.3 disclosed in NCBI. However, the present invention is not limited thereto, and any nucleic acid sequence capable of producing an amino acid with the characteristics or functions of HSP40 is encompassed. Furthermore, the amino acid sequence of HSP40 may be or comprise the sequence of NCBI Reference Sequence: AAH19827.1 or AAH02352.1 disclosed in NCBI, or may be or comprise a sequence having at least 80%, 85%, 90%, or 95% sequence identity with the sequence of NCBI Reference Sequence: AAH19827.1 or AAH02352.1. However, the present invention is not limited thereto, and any amino acid sequence exhibiting the characteristics or functions of HSP40 is included.

The “HSP60” is also referred to as ‘chaperonin (Cpn)’, and the gene encoding HSP60 may be or comprise a nucleic acid sequence that encodes the amino acid sequence of human-derived HSP60, or may be or comprise a nucleic acid sequence of NCBI Reference Sequence: NM_002156.5 or NM 199440.2 disclosed in NCBI. The nucleic acid sequence of HSP60 may be or comprise a sequence having at least 80%, 85%, 90%, or 95% sequence identity with the nucleic acid sequence of NCBI Reference Sequence: NM_002156.5 or NM_199440.2 disclosed in NCBI. However, the present invention is not limited thereto, and any nucleic acid sequence capable of producing an amino acid with the characteristics or functions of HSP60 is encompassed. Furthermore, the amino acid sequence of HSP60 may be or comprise the sequence of NCBI Reference Sequence: NP_002147.2 or NP_955472.1 disclosed in NCBI, and may be or comprise a sequence having at least 80%, 85%, 90%, or 95% sequence identity with the sequence of NCBI Reference Sequence: NP_002147.2 or NP_955472.1. However, the present invention is not limited thereto, and any amino acid sequence exhibiting the characteristics or functions of HSP60 is included.

The “HSP70” is also referred to as ‘DnaK’, and the gene encoding HSP70 may be or comprise a nucleic acid sequence that encodes the amino acid sequence of human-derived HSP70, or may be or comprise a nucleic acid sequence of NCBI Reference Sequence: NM_002154.4 or L12723.2 disclosed in NCBI. The nucleic acid sequence may be or comprise a sequence having at least 80%, 85%, 90%, or 95% sequence identity with the nucleic acid sequence of NCBI Reference Sequence: NM 002154.4 or L12723.2 disclosed in NCBI. However, the present invention is not limited thereto, and any nucleic acid sequence capable of producing an amino acid with the characteristics or functions of HSP70 is encompassed. Furthermore, the amino acid sequence of HSP70 may be or comprise the sequence of NCBI Reference Sequence: NP_002145.3 or AAA02807.1 disclosed in NCBI, and may be or comprise a sequence having at least 80%, 85%, 90%, or 95% sequence identity with the sequence of NCBI Reference Sequence: NP_002145.3 or AAA02807.1. However, the present invention is not limited thereto, and any amino acid sequence exhibiting the characteristics or functions of HSP70 is included.

The “HSP90” is also referred to as ‘HtpG’, and the gene encoding HSP90 may be or comprise a nucleic acid sequence that encodes the amino acid sequence of human-derived HSP90, or may be or comprise a nucleic acid sequence of NCBI Reference Sequence: NM_001017963.3, NM_005348.4 or BC121062.2 disclosed in NCBI. The nucleic acid sequence of HSP90 may be or comprise a sequence having at least 80%, 85%, 90%, or 95% sequence identity with the nucleic acid sequence of NCBI Reference Sequence: NM_001017963.3, NM_005348.4 or BC121062.2 disclosed in NCBI. However, the present invention is not limited thereto, and any nucleic acid sequence capable of producing an amino acid with the characteristics or functions of HSP90 is encompassed. Furthermore, the amino acid sequence of HSP90 may be or comprise the sequence of NCBI Reference Sequence: NP_001017963.2 or NP_005339.3 disclosed in NCBI, and may be or comprise a sequence having at least 80%, 85%, 90%, or 95% sequence identity with the sequence of NCBI Reference Sequence: NP_001017963.2 or NP_005339.3. However, the present invention is not limited thereto, and any amino acid sequence exhibiting the characteristics or functions of HSP90 is encompassed.

Specifically, the HVEM or its ligand inhibitor and the HSP inhibitor may target HVEM, its ligand, and/or HSP. For example, the anticancer agent may target only HVEM; only a ligand of HVEM; only HSP; both HVEM and its ligand; both HVEM and HSP; both a ligand of HVEM and HSP; or HVEM, its ligand, and HSP simultaneously.

Preferably, the HVEM or ligand inhibitor and the HSP inhibitor may include, without limitation, compounds, proteins, fusion proteins, compound-protein complexes, drug-protein complexes, antibodies, compound-antibody complexes, drug-antibody complexes, amino acids, peptides, viruses, carbohydrates, lipids, nucleic acids, extracts, or fractions. Preferably, the inhibitors may be antibodies, and more specifically, bispecific or trispecific antibodies.

As used herein, the term “inhibitor” may be used interchangeably with “suppressor” or “antagonist,” and “inhibition” may be used interchangeably with “suppression.”

Additionally, the inhibitor of HVEM or its ligand, and the HSP inhibitor, may be independently different or the same type of substance. For example, all inhibitors may be antibodies. In another example, two of the inhibitors may be antibodies and the other one may be a compound.

Specifically, the inhibitors may include, but are not limited to, compounds, peptides, peptide mimetics, fusion proteins, antibodies, aptamers, or antibody-drug conjugates (ADCs) that specifically bind to HVEM, its ligand, and/or HSP proteins.

The term “specific” refers to the ability to bind only to the target protein without affecting other proteins within the cell.

The term “antibody” includes monoclonal antibodies, polyclonal antibodies, bispecific antibodies, multispecific antibodies, chimeric antibodies, humanized antibodies, and human antibodies. It also includes known or commercially available antibodies in the relevant technical field in addition to newly developed antibodies. The antibody includes not only a full-length form comprising two heavy chains and two light chains, as long as it specifically binds to HVEM, its ligand, and/or an HSP protein, but also functional fragments of the antibody molecule. The functional fragment of the antibody molecule refers to a fragment that retains at least antigen-binding capability, and includes, but is not limited to, Fab, F(ab′), F(ab′)₂, and Fv.

The term “peptide mimetics” refers to peptides or non-peptides that inhibit the binding domain of a protein involved in the activation of HVEM, a ligand of HVEM, and/or HSP proteins.

The term “aptamer” refers to a single-stranded nucleic acid (DNA, RNA, or modified nucleic acid) that forms a stable tertiary structure by itself and binds to a target molecule with high affinity and specificity.

Additionally, the inhibitors may include antisense nucleic acids, siRNAs, shRNAs, miRNAs, or ribozymes that bind complementarily to the DNA or mRNA of HVEM, a ligand of HVEM, and/or HSP, without being limited thereto.

The term “antisense nucleic acid” refers to DNA, RNA, or a fragment or derivative thereof that contains a nucleic acid sequence complementary to a particular mRNA sequence. It functions to inhibit translation of the mRNA into protein by binding or hybridizing complementarily to the mRNA sequence.

The term “small interfering RNA (siRNA)” refers to a short double-stranded RNA that may induce RNA interference (RNAi) by cleaving a specific mRNA. It comprises a sense RNA strand having a sequence homologous to the mRNA of a target gene and an antisense RNA strand having a sequence complementary thereto. Since siRNA may suppress the expression of the target gene, it is used in methods such as gene knockdown or gene therapy.

The term “short hairpin RNA (shRNA)” refers to a single-stranded RNA that is divided into a stem region forming a double-stranded portion through hydrogen bonding and a loop region having a loop structure. It is processed by proteins such as Dicer into siRNA and may perform the same functions as siRNA.

The term “micro RNA (miRNA)” refers to 21 to 23 non-coding RNA that regulate gene expression post-transcriptionally by promoting degradation of or inhibiting translation of target RNAs.

The term “ribozyme” refers to an RNA molecule having an enzyme-like function that recognizes a specific nucleotide sequence and cleaves it by itself. It is composed of a region that binds specifically to a target messenger RNA strand through a complementary nucleotide sequence, and a region that cleaves the target RNA.

These antisense nucleic acids, siRNAs, shRNAs, miRNAs, and ribozymes that complementarily bind to the DNA or mRNA of HVEM, a ligand of HVEM, and/or HSP may inhibit essential activities involved in various biological functions of HVEM, a ligand of HVEM, and/or HSP, including but not limited to transcription, cytoplasmic translocation, maturation, or translation. The pharmaceutical composition of the present invention, which comprises an effective amount of an inhibitor of HVEM or its ligand, and an HSP inhibitor, may be administered to a subject in need of cancer prevention or treatment.

The term “prevention” refers to any action that inhibits or delays the onset of cancer by administration of the pharmaceutical composition of the present invention. The term “treatment” refers to any action that improves or cures cancer by administration of the composition.

The cancers that may be prevented or treated by the pharmaceutical composition of the present invention include, but not limited to, biliary tract cancer, gastric cancer, lung cancer, liver cancer, colorectal cancer, colon cancer, small intestine cancer, pancreatic cancer, brain cancer, bone cancer, melanoma, breast cancer, adenosis, uterine cancer, cervical cancer, head and neck cancer, esophageal cancer, thyroid cancer, parathyroid cancer, kidney cancer, sarcoma, prostate cancer, urethral cancer, bladder cancer, hematological cancer, leukemia, lymphoma, and fibroadenoma. The effective amount may be a “therapeutically effective amount” or a “prophylactically effective amount”. The term “therapeutically effective amount” refers to any amount of a drug or therapeutic agent which, when administered alone or in combination with other therapeutic agents, results in a reduction in the severity of disease symptoms, an increase in the frequency or duration of symptom-free periods, or the prevention of impairment or disability caused by the disease. The term “prophylactically effective amount” refers to any amount that suppresses the onset or recurrence of cancer in a subject. The level of the effective amount may be determined based on factors such as the severity of the condition, age, sex, activity of the drug, sensitivity to the drug, timing and route of administration, rate of excretion, duration of treatment, concomitant medications, and other factors well known in the medical field.

The term “administration” refers to the physical introduction of a composition into a subject using any method or delivery system known to those skilled in the art. The route of administration for the pharmaceutical composition of the present invention includes, but not limited to, oral administration, or parenteral routes such as intravenous, intramuscular, subcutaneous, intraperitoneal, spinal, or other non-oral routes, for example, via injection or infusion. The frequency of administration of the composition of the present invention may be, for example, a single administration, multiple administrations, or administration over one or more extended periods.

The pharmaceutical composition may be administered in different dosages depending on the subject's age, sex, or weight. Specifically, the composition may be administered at 0.1 to 100 mg/kg once daily or multiple times per day, or at intervals of several days to several months, depending on the symptoms. The dosage may be adjusted depending on the administration route, disease severity, sex, weight, age, and other factors.

The composition may further comprise pharmaceutically acceptable carriers, excipients, or diluents commonly used in the preparation of pharmaceutical composition. For example, it includes, but not limited to, lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methyl hydroxybenzoate, propyl hydroxybenzoate, talc, magnesium stearate, and mineral oil.

The term “subject” includes humans and non-human animals. Non-human animals may be vertebrates such as primates, dogs, cattle, horses, pigs, or rodents such as mice, rats, and guinea pigs. As used herein, the term “subject” is used interchangeably with “individual” and “patient”. The HVEM or its ligand inhibitor and HSP inhibitor comprised in the pharmaceutical composition may be formulated for simultaneous, sequential, or separate administration. For example, they may be administered simultaneously in a single formulation, or administered simultaneously, sequentially, or separately as separate formulations. To allow for simultaneous, sequential, or separate administration, the HVEM or its ligand inhibitor and the HSP inhibitor comprised in the pharmaceutical composition of the present invention may be formulated separately in individual containers, or together in a single container. In addition, the HVEM or its ligand inhibitor and the HSP inhibitor comprised in the pharmaceutical composition of the present invention may have the same or different therapeutically effective amounts, administration times, administration intervals, routes of administration, or treatment durations.

The pharmaceutical composition may also be administered in combination with other therapeutic agents. In such cases, the composition and the other therapeutic agents may be administered simultaneously, sequentially, or separately. The other therapeutic agents may include drugs such as compounds or proteins with effects on cancer prevention, treatment, or improvement, without being limited thereto.

Furthermore, the pharmaceutical composition may be formulated for simultaneous, sequential, or separate administration with other therapeutic agents. For example, the HVEM or its ligand inhibitor and the HSP inhibitor, together with other therapeutic agents, may be administered in a single formulation, or as separate formulations administered simultaneously, sequentially, or separately. To allow for simultaneous, sequential, or separate administration, the HVEM or ligand inhibitor, the HSP inhibitor, and other therapeutic agents comprised in the pharmaceutical composition of the present invention may be formulated separately in individual containers, or together in a single container. In addition, the HVEM or ligand inhibitor, the HSP inhibitor, and other therapeutic agents comprised in the pharmaceutical composition of the present invention may have the same or different therapeutically effective amounts, administration times, administration intervals, routes of administration, or treatment durations.

Another aspect of the present invention provides a method for preventing or treating cancer, comprising administering an inhibitor of HVEM or its ligand, and an HSP inhibitor to a subject in need of prevention or treatment of cancer.

Unless otherwise specified, each term used in the method for cancer prevention or treatment according to the present invention has the same meaning as described in the pharmaceutical composition for preventing or treating cancer.

In the method for cancer prevention or treatment of the present invention, the HVEM or ligand inhibitor and HSP inhibitor may be administered simultaneously, sequentially, or individually to the subject.

Additionally, the HVEM or ligand inhibitor and the HSP inhibitor may be administered simultaneously, sequentially, or individually with other therapeutic agents to the subject.

The term “simultaneous” administration refers to the administration, at the same time, of the HVEM or ligand inhibitor and the HSP inhibitor in the form of a single formulation, or the administration of the HVEM or ligand inhibitor, the HSP inhibitor, and other therapeutic agents in the form of a single formulation. It also refers to the administration, at the same time, of the HVEM or ligand inhibitor and the HSP inhibitor as separate formulations, or the administration of the HVEM or ligand inhibitor, the HSP inhibitor, and other therapeutic agents as separate formulations, wherein the routes of administration of the HVEM or ligand inhibitor, HSP inhibitor, and/or other therapeutic agents may be different.

The term “sequential” administration refers to administering the HVEM or ligand inhibitor and the HSP inhibitor, or the HVEM or ligand inhibitor, the HSP inhibitor, and other therapeutic agents in a relatively continuous manner, allowing only the minimum necessary time between administrations.

The term “separate” or “individual” administration refers to administering the HVEM or ligand inhibitor and the HSP inhibitor, or the HVEM or ligand inhibitor, the HSP inhibitor, and other therapeutic agents with a defined time interval between each. The specific method of administration may be appropriately selected by a physician or expert based on therapeutic efficacy and side effects.

Another aspect of the present invention provides a method for screening anticancer agents, comprising: (a) treating a biological sample isolated from a subject suffering from cancer or a cancer disease animal model with a candidate inhibitor of HVEM or a candidate inhibitor of a ligand of HVEM, and a candidate inhibitor of HSP; (b) analyzing anticancer activity in the group treated with the candidate inhibitors in step (a); and (c) determining the candidate inhibitor of HVEM or the candidate inhibitor of a ligand of HVEM, and the candidate inhibitor of HSP as anticancer agents, when the anticancer activity analyzed in step (b) is increased compared to that of a control group.

Unless otherwise specified, each term used in the method for screening anticancer agents has the same meanings as previously described.

The term “candidate inhibitor of HVEM” as used in the present invention refers to a substance that is expected to inhibit or suppress the activity of HVEM, and may include, without limitation, a substance that is expected to directly binds to HVEM, a substance that is expected to directly binds to a ligand of HVEM, or a substance that is expected to inhibit the binding of HVEM to its ligand. Such substances may include antibodies, compounds, genes, or proteins.

The term “candidate inhibitor of a ligand of HVEM” as used in the present invention refers to a substance that is expected to inhibit or suppress the activity of a ligand of HVEM, and may include, without limitation, a substance that is expected to directly binds to the ligand of HVEM or a substance that is expected to inhibit the binding of the ligand to HVEM. Such substances may include antibodies, compounds, genes, or proteins.

The term “candidate inhibitor of HSP” as used in the present invention refers to a substance that is expected to inhibit or suppress the activity of HSP, and may include, without limitation, a substance that is expected to directly binds to HSP, a substance that is expected to directly binds to a ligand of HSP, or a substance that is expected to inhibit the binding of HSP to its ligand. Such substances may include antibodies, compounds, genes, or proteins.

In the present invention, the HVEM inhibitor candidate, the HVEM ligand inhibitor candidate, or the HSP inhibitor candidate may be an anticancer agent candidate. That is, the inhibitor candidate refers to a substance expected to prevent or treat cancer, and may include, without limitation, a substance that is expected to directly or indirectly prevent, treat, alleviate, or improve cancer. Such substances may include antibodies, compounds, genes, or proteins.

The term “biological sample” as used in the present invention may be a cell, tissue, blood, or an organoid derived therefrom, but is not limited thereto. The biological sample may be treated with the inhibitor candidate in either a manipulated or unmanipulated state. In addition, the biological sample may be a cancer organoid comprising cancer cells.

In the present invention, the control group may be a group not treated with the HVEM inhibitor candidate or the HVEM ligand inhibitor candidate and the HSP inhibitor candidate, a group treated with only the HVEM inhibitor candidate or the HVEM ligand inhibitor candidate, or a group treated with only the HSP inhibitor candidate. Optionally, the control group may be a substance known to be effective in preventing or treating cancer.

Specifically, step (a) is a step of treating cancer cells, cancer tissue, blood, or cancer organoids isolated from a subject suffering from cancer, or a cancer disease animal model, with the inhibitor candidate. The HVEM inhibitor candidate or HVEM ligand inhibitor candidate and the HSP inhibitor candidate may be administered simultaneously, continuously, or sequentially, and such administration may be carried out using methods known to those skilled in the art. For example, the inhibitor candidates may be treated by co-culturing with cancer cells or cancer organoids, or by administering the candidates into an in vivo system containing cancer cells, but the method is not limited thereto, and one skilled in the art may use a method suitable for the purpose of the present invention. In addition, the HSP inhibitor candidate may be treated after treating the HVEM inhibitor candidate or HVEM ligand inhibitor candidate, or the HVEM inhibitor candidate or HVEM ligand inhibitor candidate may be treated after treating the HSP inhibitor candidate.

In addition, step (b) is a step of analyzing the anticancer activity of the inhibitor candidate, which may be a step of analyzing the increase in cancer cell death, reduction in tumor size, or reduction in tumor weight. The analysis may be performed using any method known to those skilled in the art. For example, Western blot, co-immunoprecipitation assay, enzyme-linked immunosorbent assay (ELISA), tissue immunostaining, fluorescence activated cell sorter (FACS), and tissue biopsy analysis may be used, but not limited thereto, and one skilled in the art may use a method appropriate for the purpose of the present invention.

Finally, step (c) is a step of determining whether the inhibitor candidate may be used as an anticancer agent. If the HVEM inhibitor candidate or HVEM ligand inhibitor candidate and the HSP inhibitor candidate increase cancer cell death, reduce tumor size, or reduce tumor weight, they may be determined to be usable for the prevention or treatment of cancer.

The combination of an inhibitor of HVEM or its ligand, and an HSP inhibitor exhibits a synergistic effect on anticancer activity compared to when each is used alone, and demonstrates efficacy within a short time of 72 hours post-administration. Therefore, the combination of an HVEM or ligand inhibitor and an HSP inhibitor can be usefully applied to the prevention or treatment of cancer.

### BRIEF DESCRIPTION OF FIGURES

FIG. 1 is a graph showing the results of treating colorectal cancer organoids with various concentrations of an HVEM inhibitor. A shows the growth rate (%) of the cancer organoids over time following HVEM inhibitor treatment, and B shows the death rate (%) of the cancer organoids according to HVEM inhibitor concentration. ‘IgG1’ indicates the control single antibody, and ‘HVEM’ indicates the HVEM inhibitor.

FIG. 2 is a graph showing the results of treating colorectal cancer organoids with various concentrations of the HSP inhibitor AUY922. A relates to the growth rate (%) of cancer organoids according to the treatment duration of the HSP inhibitor, and B relates to the death rate (%) of cancer organoids according to the treatment concentration of the HSP inhibitor. “AUY922” refers to an HSP90 inhibitor.

FIG. 3 is a graph showing the synergistic effect on cancer organoid death resulting from the combination treatment of an HVEM inhibitor and an HSP inhibitor AUY922, assessed 72 hours after treatment. A relates to colorectal cancer organoids, and B relates to lung cancer organoids. “HVEM” refers to an HVEM inhibitor, and “AUY922” refers to an HSP90 inhibitor.

FIG. 4 is a graph showing the synergistic effect on cancer organoid death resulting from the combination treatment of a BTLA inhibitor and an HSP inhibitor AUY922, assessed 72 hours after treatment. A relates to colorectal cancer organoids, and B relates to lung cancer organoids. “BTLA” refers to a BTLA inhibitor, and “AUY922” refers to an HSP90 inhibitor.

FIG. 5 is a graph showing the synergistic effect on cancer organoid death resulting from the combination treatment of an HVEM inhibitor with an HSP70 inhibitor, an HVEM inhibitor with an HSP90 inhibitor, a BTLA inhibitor with an HSP70 inhibitor, and a BTLA inhibitor with an HSP90 inhibitor, assessed 72 hours after treatment. It shows the cancer organoid death rates (%) under single-agent and combination treatment conditions. A relates to pancreatic cancer organoids, and B relates to biliary tract cancer organoids. “VER-155008” refers to an HSP70 inhibitor, and “AUY922” refers to an HSP90 inhibitor.

FIG. 6 is a graph showing the synergistic effect of combination treatment of an HVEM inhibitor with various HSP family protein inhibitors, assessed 72 hours after treatment. It shows the colorectal cancer organoid death rates (%) under single-agent and combination treatment conditions. “IgG1” refers to a control single antibody, “HVEM” refers to an HVEM inhibitor, “AUY922” refers to an HSP90 inhibitor, “STA-9090” refers to an HSP90 inhibitor, “17-AAG” refers to an HSP90 inhibitor, “BIIB021” refers to an HSP90 inhibitor, “AT13387” refers to an HSP90 inhibitor, “HSP990” refers to an HSP90 inhibitor, “KNK437” refers to an HSP40 inhibitor, and “VER-155008” refers to an HSP70 inhibitor.

FIG. 7 is a graph showing the synergistic effect on in vivo cancer cell death resulting from the combination treatment of an HVEM inhibitor and an HSP inhibitor AUY922, or a BTLA inhibitor and an HSP inhibitor AUY922, in terms of changes in tumor weight. “IgG” refers to a control single antibody, “HVEM” refers to an HVEM inhibitor, “BTLA” refers to a BTLA inhibitor, and “AUY922” refers to an HSP90 inhibitor.

FIG. 8 shows tumor images collected at the end of the experiment to demonstrate the synergistic effect on in vivo cancer cell death resulting from the combination treatment of an HVEM inhibitor and an HSP inhibitor AUY922, or the combination of an HVEM inhibitor, a BTLA inhibitor, and an HSP inhibitor AUY922. “IgG” refers to a control single antibody, “HVEM” refers to an HVEM inhibitor, “BTLA” refers to a BTLA inhibitor, and “AUY922” refers to an HSP90 inhibitor.

FIG. 9 is a graph showing the synergistic effect on in vivo cancer cell death resulting from the combination treatment of an HVEM inhibitor and an HSP inhibitor AUY922, or the combination of an HVEM inhibitor, a BTLA inhibitor, and an HSP inhibitor AUY922, in relation to the weight of tumors collected at the end of the experiment. “IgG” refers to a control single antibody, “HVEM” refers to an HVEM inhibitor, “BTLA” refers to a BTLA inhibitor, and “AUY922” refers to an HSP90 inhibitor.

FIG. 10 is a graph showing the synergistic effect on in vivo cancer cell death resulting from the combination treatment of an HVEM inhibitor and an HSP inhibitor AUY922, or the combination of an HVEM inhibitor, a BTLA inhibitor, and an HSP inhibitor AUY922, in relation to the presence of CD8+ T cells infiltrated into tumor tissues collected at the end of the experiment. “IgG” refers to a control single antibody, “HVEM” refers to an HVEM inhibitor, “BTLA” refers to a BTLA inhibitor, and “AUY922” refers to an HSP90 inhibitor.

### EXAMPLES

Hereinafter, the present invention will be described in further detail with reference to Examples. These Examples are provided to illustrate the invention more concretely, and the scope of the invention is not limited thereto.

### Example 1. In Vitro Anticancer Effect of Combination Treatment with HVEM Inhibitor and HSP Inhibitor

To evaluate whether the combination of HVEM inhibitor and HSP inhibitor exerts a synergistic effect as a cancer preventive or therapeutic agent, we conducted the following experiment. Specifically, colorectal cancer organoids were treated with either an HVEM inhibitor alone, an HSP inhibitor alone, or a combination of both. The anticancer efficacy of the combination treatment was assessed by measuring cancer organoid viability.

Organoids are organ-like structures produced by three-dimensional culturing of cells derived from tissues or organs. Organoids have the advantages of being suitable for long-term culture and cryopreservation, as well as being easy to manipulate and observe. Furthermore, since immortalization is not required, the original characteristics of the cells are preserved, and they can reproduce the hierarchical and histological structures of cells that were observable only in vivo. Therefore, organoids serve as experimental models that enable the study of physiological phenomena at a higher level than cell models. Due to these characteristics, organoids allow for more accurate drug evaluation compared to immortalized cell lines, in which intrinsic cellular properties are altered, or animal models that differ structurally from the human body. In particular, since organoids are derived from patient tissues, they offer the advantage of enabling the assessment of not only drug safety but also efficacy prior to clinical application in humans.

For HVEM inhibitor, anti-HVEM antibodies (CW10, Santa Cruz, #sc-21718; R&D Systems, #MAB356-SP) were used at 10 nM. For inhibitor of HVEM ligands, anti-BTLA antibodies (Mybiosource, #MBS633646; Adipogen, #AG-20B-0049) were used at 10 nM. For HSP inhibitors, Luminespib (AUY922, Selleck, #s1069; HSP90-targeting), Ganetespib (STA-9090, Selleck, #s1159; HSP90-targeting), Tanespimycin (17-AAG, Selleck, #s1141; HSP90-targeting), BIIB021 (Selleck, #s1175; HSP90-targeting), Onalespib (AT13387, Selleck, #s1163; HSP90-targeting), HSP990 (#s7097; Selleck, HSP90-targeting), KNK437 (#s7750; Selleck, HSP40-targeting), and VER-155008 (Selleck, #s7751; HSP70-targeting) were used.

The viability of cancer organoids was assessed through growth rate and death rate of cancer organoids as follows.

$$Cancer organoid death rate ( \% ) = [ \text{} 1 - \text{} ( Growth \text{} rate \text{} of \text{} cancer \text{} organoids in the inhibitor - treated group \text{*)} \text{} / \text{} ( Growth \text{} rate \text{} of \text{} cancer organoids in the untreated control group \text{**)} ] \times 100 * Growth rate of cancer organoids \text{} in \text{} the \text{} inhibitor \text{} - \text{} treated \text{} group ( \% ) = \text{} ( Area of cancer organoids at 24 , \text{} 48 , \text{} or 72 hours after inhibitor treatment ) \text{} / \text{} ( Area of cancer organoids at 0 hours ) \times 100 ** Growth rate of cancer organoids \text{} in \text{} the \text{} untreated \text{} control \text{} group \text{} ( \text{} \% ) = \text{} ( Area of cancer \text{} organoids at 24 , \text{} 48 , \text{} or 72 hours without inhibitor \text{} treatment ) \text{} / \text{} ( Area of cancer organoids at 0 hours ) \times 100$$

As described above, the synergistic effect of the combination treatment with an HVEM inhibitor and an HSP inhibitor on cancer therapy was analyzed and is described in Examples 1.1 to 1.5 below.

### Example 1.1. Concentration-Dependent Effect of HVEM Inhibitor

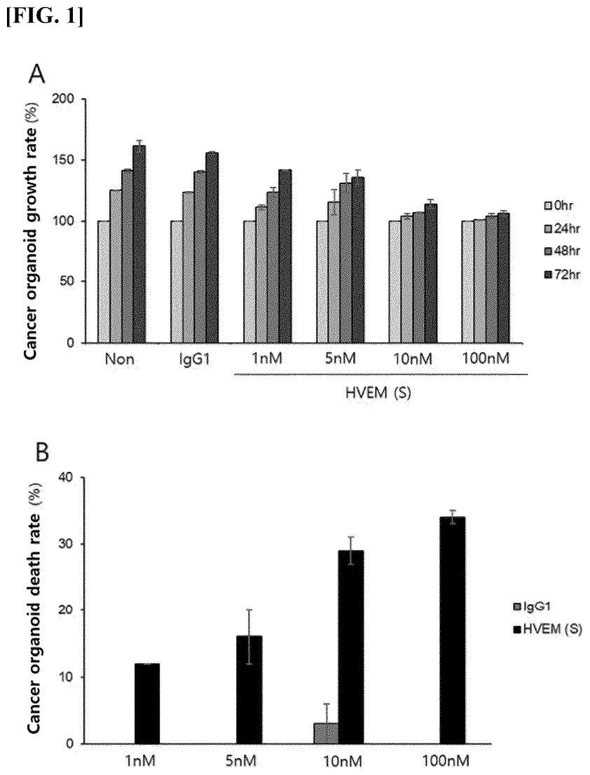

As shown in FIG. 1, treatment of colorectal cancer organoids with increasing concentrations of HVEM inhibitor led to decreased organoid growth over time (FIG. 1A) and increased colorectal cancer organoids death rate (FIG. 1B). In addition, it was confirmed that treatment with a high concentration (10 nM or higher) of the HVEM inhibitor resulted in more than a 2.8-fold increase in cancer organoid death rate compared to treatment with a low concentration (1 to 5 nM). In particular, when administered at the same concentration, the HVEM inhibitor exhibited approximately 10 times greater efficacy compared to the control antibody IgG1 alone.

### Example 1.2. Concentration-Dependent Effect of HSP Inhibitor

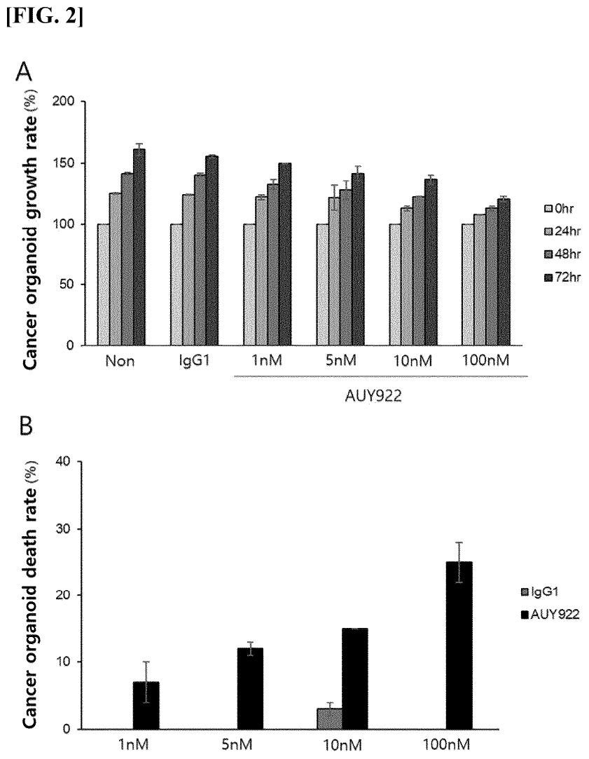

As shown in FIG. 2, colorectal cancer organoids treated with increasing concentrations of HSP90 inhibitor showed reduced growth over time (FIG. 2A) and increased cancer organoids death rate (FIG. 2B). In addition, it was confirmed that treatment with a high concentration (100 nM or higher) of the HSP90 inhibitor resulted in more than a 3.6-fold increase in cancer organoid death rate compared to treatment with a low concentration (1 to 5 nM).

### Example 1.3. Synergistic Effect of HVEM Inhibitor and HSP90 Inhibitor

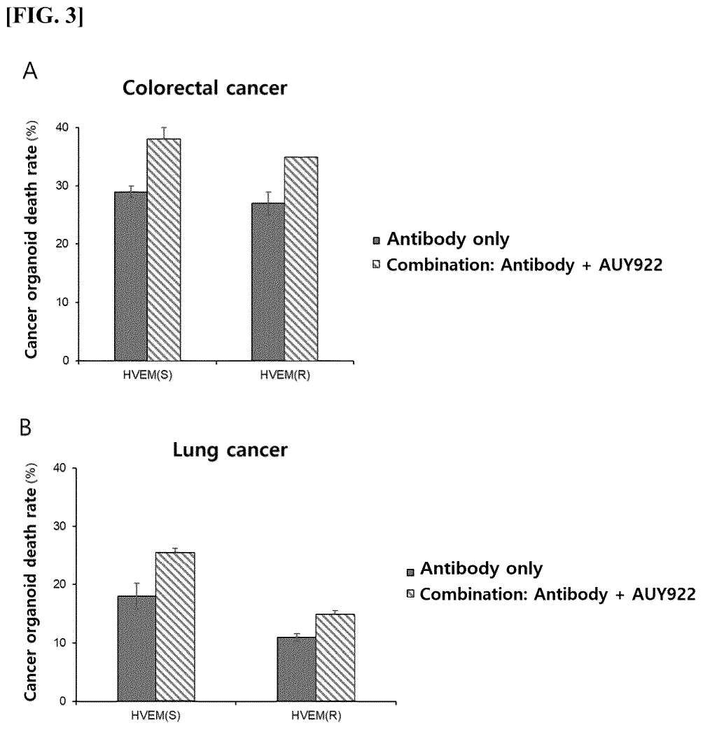

As shown in FIG. 3A, it was confirmed that the combination treatment of an HVEM inhibitor and an HSP90 inhibitor resulted in an increased cancer organoid death rate in colorectal cancer organoids, compared to treatment with the HVEM inhibitor alone. Specifically, a strong synergistic effect was observed regardless of the manufacturer, such as the HVEM inhibitor from Santa Cruz or the HVEM inhibitor from R&D Systems, and the combination treatment resulted in approximately a 1.5-fold increase in cancer organoid death rate compared to treatment with the HVEM inhibitor alone.

As shown in FIG. 3B, it was also confirmed that, in lung cancer organoids, the combination treatment of an HVEM inhibitor and an HSP90 inhibitor resulted in an increased cancer organoid death rate compared to treatment with the HVEM inhibitor alone. Specifically, a strong synergistic effect was observed regardless of the manufacturer, such as the HVEM inhibitor from Santa Cruz or the HVEM inhibitor from R&D Systems, and the combination treatment resulted in an approximately 2.1- to 3.7-fold increase in cancer organoid death rate compared to treatment with the HVEM inhibitor alone.

These results demonstrate that the combination of an HVEM inhibitor and an HSP90 inhibitor exerts a strong synergistic effect on cancer organoid death, particularly by effectively targeting and killing cancer cells within the tumor microenvironment. Therefore, it can be effectively used for the prevention or treatment of various cancers, including colorectal and lung cancer.

### Example 1.4. Synergistic Effect of Combination Treatment with a BTLA Inhibitor and an HSP Inhibitor

To further verify the synergistic effect of the combination treatment with an HVEM inhibitor and an HSP90 inhibitor, the synergistic effect with a BTLA inhibitor, which is an inhibitor of the ligand of HVEM, was evaluated.

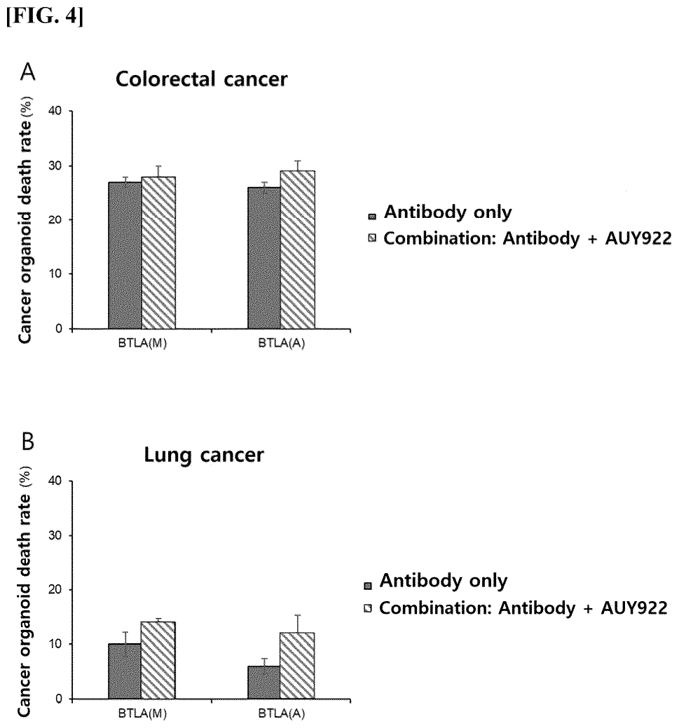

As shown in FIG. 4A, it was confirmed that the combination treatment of a BTLA inhibitor and an HSP90 inhibitor resulted in an increased cancer organoid death rate in colorectal cancer organoids, compared to treatment with the BTLA inhibitor alone. Specifically, a strong synergistic effect was observed regardless of the manufacturer, such as the BTLA inhibitor from Mybiosource or the BTLA inhibitor from Adipogen, and the combination treatment resulted in more than a 1.6-fold increase in cancer organoid death rate compared to treatment with the BTLA inhibitor alone. As shown in FIG. 4B, it was also confirmed that, in lung cancer organoids, the combination treatment of a BTLA inhibitor and an HSP90 inhibitor resulted in an increased cancer organoid death rate compared to treatment with the BTLA inhibitor alone. Specifically, a strong synergistic effect was observed regardless of the manufacturer, such as the BTLA inhibitor from Mybiosource or the BTLA inhibitor from Adipogen, and the combination treatment resulted in approximately a 1.7- to 2.0-fold increase in cancer organoid death rate compared to treatment with the BTLA inhibitor alone.

These results demonstrate that the combination of a BTLA inhibitor and an HSP90 inhibitor exerts a strong effect on cancer organoid death, particularly by effectively targeting and killing cancer cells within the tumor microenvironment. Therefore, it can be effectively used for the prevention or treatment of various cancers, including colorectal and lung cancer.

### Example 1.5. Synergistic Effect of Combination Treatment with an HVEM Inhibitor and HSP Family Inhibitors

As demonstrated in Examples 1.3 and 1.4, a synergistic effect on cancer organoid death was observed when an HVEM inhibitor was used in combination with an HSP90 inhibitor, compared to the use of the HVEM inhibitor alone. Accordingly, in the present example, it was investigated whether inhibitors of other HSP family proteins, in addition to HSP90, also exhibit synergistic effects when used in combination with an HVEM inhibitor.

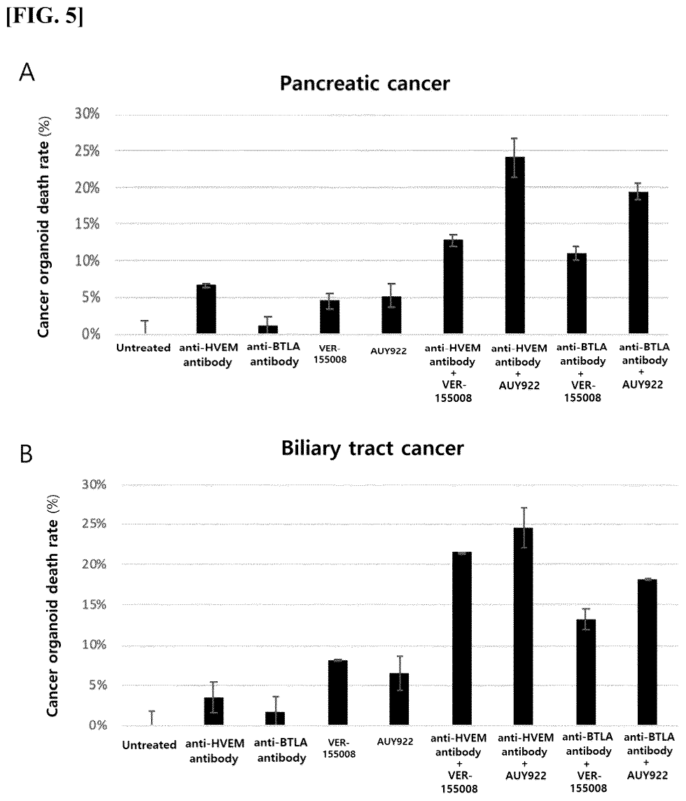

As shown in FIG. 5A, in pancreatic cancer organoids, compared to the monotherapy with HVEM inhibitor (anti-HVEM antibody), BTLA inhibitor (anti-BTLA antibody), HSP70 inhibitor (VER-155008), or HSP90 inhibitor (AUY922), the combination of HVEM inhibitor and HSP70 inhibitor showed approximately a 2-fold increase in efficacy, and compared to the monotherapy with HVEM inhibitor or HSP90 inhibitor, the combination of HVEM inhibitor and HSP90 inhibitor showed approximately a 5-fold increase in efficacy. In addition, compared to the monotherapy with BTLA inhibitor or HSP70 inhibitor, the combination of BTLA inhibitor and HSP70 inhibitor showed approximately a 2-fold increase in efficacy, and compared to the monotherapy with BTLA inhibitor or HSP90 inhibitor, the combination of BTLA inhibitor and HSP90 inhibitor showed approximately a 3-fold increase in efficacy. This synergistic effect was also commonly observed in biliary tract organoids, as shown in FIG. 5B.

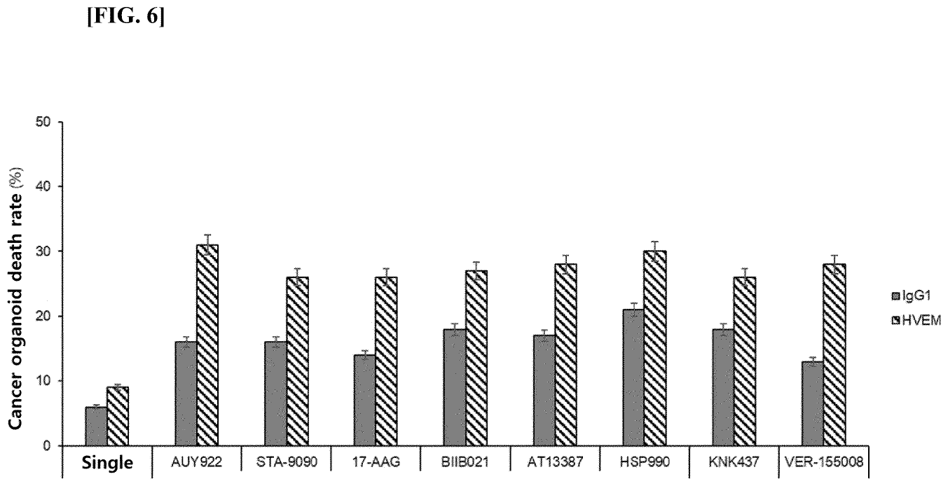

As shown in FIG. 6, it was confirmed that the synergistic effect with the HVEM inhibitor was similarly observed across inhibitors targeting different types of HSP family proteins, including HSP90, HSP40, and HSP70. Specifically, compared to treatment with the HVEM inhibitor alone, the combination of the HVEM inhibitor and HSP90 inhibitors showed an increase in efficacy of approximately 2.9-fold (STA-9090 or 17-AAG) to 3.4-fold or more (AUY922), and this effect was commonly observed in all HSP90 inhibitors, despite their different chemical structures. In addition, compared to the HVEM inhibitor alone, the combination of the HVEM inhibitor and an HSP40 inhibitor showed an increase in cancer organoid death efficacy of approximately 2.9-fold or more. Furthermore, compared to the HVEM inhibitor alone, the combination of the HVEM inhibitor and an HSP70 inhibitor showed an increase in cancer organoid death efficacy of approximately 3.1-fold or more.

These results indicate that the combination of a BTLA inhibitor and an HSP90 inhibitor exhibits a high effect on cancer organoid death, and that excellent synergistic effects are also observed with inhibitors targeting different HSP family proteins, such as HSP90, HSP70, and HSP40. Therefore, such combinations may be usefully applied for the prevention or treatment of cancer.

### Example 2. In Vivo Anticancer Effect of Combination Treatment with HVEM Inhibitor and HSP Inhibitor

To determine whether the combination of an HVEM inhibitor and an HSP inhibitor exhibits a synergistic effect in vivo as a preventive or therapeutic agent for cancer, in vivo studies were conducted.

Specifically, monotherapy with an HVEM inhibitor, monotherapy with a BTLA inhibitor, monotherapy with an HSP inhibitor, combination therapy with an HVEM inhibitor and an HSP inhibitor, combination therapy with a BTLA inhibitor and an HSP inhibitor, or combination therapy with an HVEM inhibitor, a BTLA inhibitor, and an HSP inhibitor was administered to a mouse tumor xenograft model using colorectal cancer organoids. In addition, since the effect of inhibitors of HVEM and its ligand requires the presence of immune cells, PBMCs were introduced into the mice bearing the xenograft tumors to generate humanized mice. The inhibitors were administered a total of four times on days 15, 18, 21, and 24 after tumor transplantation.

As an HVEM (Herpesvirus entry mediator) inhibitor, an anti-HVEM antibody (CW10, Santa Cruz, #sc-21718) was used at a dose of 1 mg/kg; as an inhibitor of the HVEM ligand, an anti-BTLA antibody (Mybiosource, #MBS633646) was used at a dose of 1 mg/kg; and as an HSP (heat shock protein) inhibitor, Luminespib (AUY922, Selleck, #s1069; targeting HSP90) was used at a dose of 0.5 mg/kg.

From day 7 after tumor implantation, tumor size was measured three times per week. After completion of the experiment, the shape of the harvested tumors was analyzed, and tumor weight was measured. In addition, IHC (hCD8) staining was performed on the harvested tumor tissues to determine the presence of hCD8+ T cells infiltrating into the tissues.

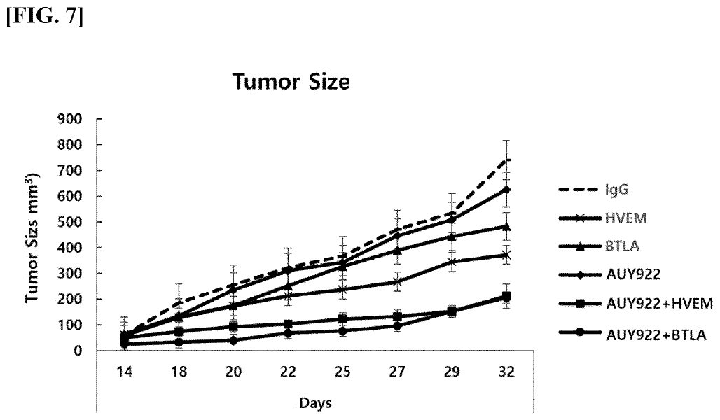

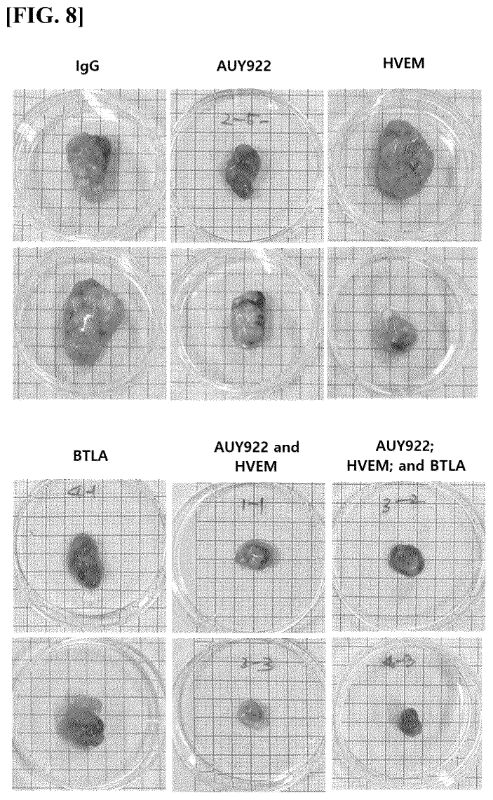

As shown in FIGS. 7 and 8, compared to the monotherapy with an HVEM inhibitor, a BTLA inhibitor, or an HSP90 inhibitor, the combination treatment of an HVEM inhibitor and an HSP90 inhibitor, a BTLA inhibitor and an HSP90 inhibitor, or an HVEM inhibitor, a BTLA inhibitor, and an HSP90 inhibitor resulted in a significant reduction in tumor size.

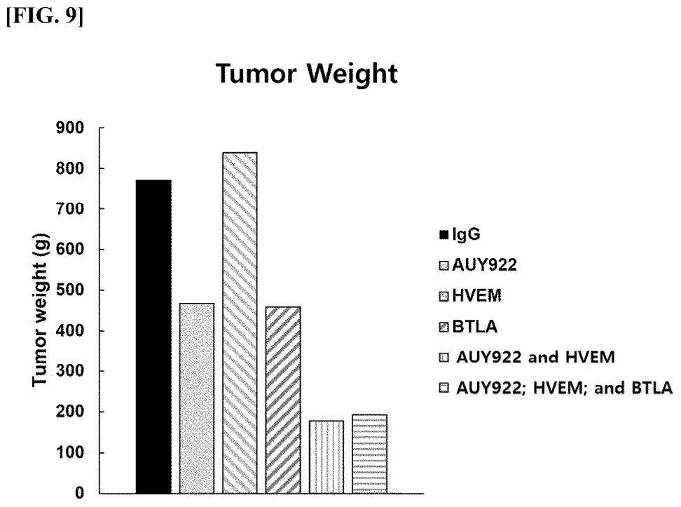

In addition, as shown in FIG. 9, compared to the monotherapy with an HVEM inhibitor, a BTLA inhibitor, or an HSP90 inhibitor, the combination treatment of an HVEM inhibitor and an HSP90 inhibitor, or the combination of an HVEM inhibitor, a BTLA inhibitor, and an HSP90 inhibitor resulted in a significant reduction in tumor weight, by approximately 2- to 3-fold or more.

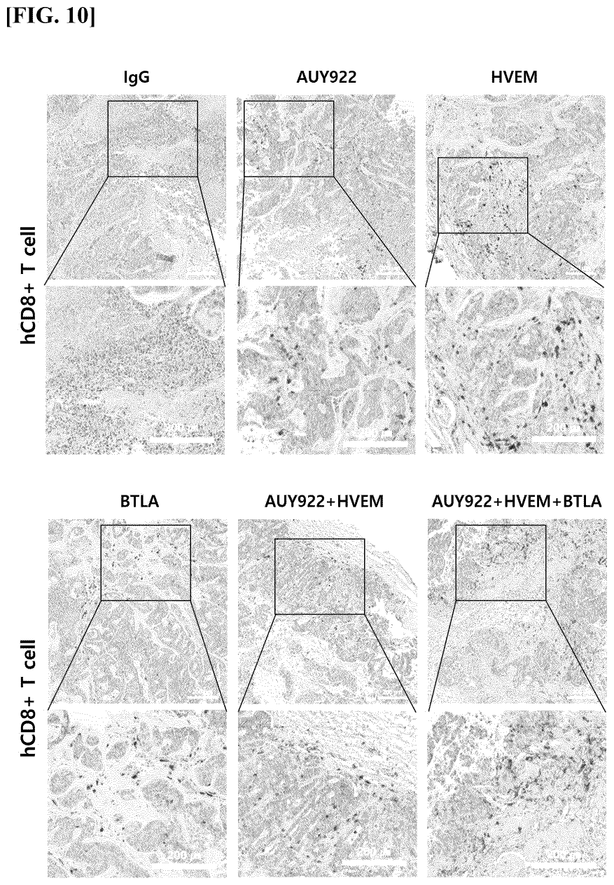

Furthermore, as shown in FIG. 10, hCD8+ T cells infiltrated into the tumor tissue were barely detected in the groups treated with an HVEM inhibitor, a BTLA inhibitor, or an HSP90 inhibitor alone. However, in the groups treated with a combination of an HVEM inhibitor and an HSP90 inhibitor, or a combination of an HVEM inhibitor, a BTLA inhibitor, and an HSP90 inhibitor, infiltration of hCD8+ T cells into the tumor tissue was observed.

These results indicate that the combination administration of an HVEM inhibitor or a BTLA inhibitor with an HSP inhibitor exhibits a high in vivo cancer cell killing effect. In particular, the effect of targeting and killing cancer cells within the tumor microenvironment was remarkably strong, suggesting that this approach can be effectively applied for the prevention or treatment of various cancers, including colorectal cancer and lung cancer.

What is claimed is:

1. A pharmaceutical composition for preventing or treating cancer, comprising (a) an inhibitor of Herpesvirus Entry Mediator (HVEM) or its ligand; and (b) an inhibitor of Heat Shock Protein (HSP).

2. The pharmaceutical composition according to claim 1, wherein the HSP is one or more selected from the group consisting of HSP40, HSP60, HSP70 and HSP90.

3. The pharmaceutical composition according to claim 1, wherein the inhibitor is one or more selected from the group consisting of a compound, a peptide, a peptide mimetic, a fusion protein, an antibody, an aptamer and an antibody-drug conjugate (ADC), which specifically binds to an HVEM or an HSP protein.

4. The pharmaceutical composition according to claim 1, wherein the inhibitor is one or more selected from the group consisting of an antisense nucleic acid, a siRNA, a shRNA, a miRNA and a ribozyme, which complementarily binds to DNA or mRNA of HVEM or HSP.

5. The pharmaceutical composition according to claim 1, wherein the cancer is one or more selected from the group consisting of biliary tract cancer, gastric cancer, lung cancer, liver cancer, colorectal cancer, colon cancer, small intestine cancer, pancreatic cancer, brain cancer, bone cancer, melanoma, breast cancer, adenosis, uterine cancer, cervical cancer, head and neck cancer, esophageal cancer, thyroid cancer, parathyroid cancer, kidney cancer, sarcoma, prostate cancer, urethral cancer, bladder cancer, hematological cancer, lymphoma and fibroadenoma.

6. A method for screening anticancer agents, comprising:
(a) treating a biological sample isolated from a subject suffering from cancer or a cancer disease animal model with a candidate inhibitor of HVEM or a candidate inhibitor of HVEM ligand, and a candidate inhibitor of HSP;
(b) analyzing anticancer activity in the group treated with the candidate inhibitors in step (a); and
(c) determining the candidate inhibitor of HVEM or the candidate inhibitor of HVEM ligand, and the candidate inhibitor of HSP as anticancer agents, when the anticancer activity analyzed in step (b) is increased compared to that of a control group.

7. The method according to claim 6, wherein in step (a), the candidate inhibitor of HVEM or the candidate inhibitor of HVEM ligand, and the candidate inhibitor of HSP are administered simultaneously, continuously, or sequentially.

8. The method according to claim 6, wherein the biological sample is cells, tissues, blood, or organoids derived therefrom.

9. The method according to claim 6, wherein the anticancer activity in step (b) is an increased cancer cell death, a reduced tumor size, or a reduced tumor weight.

10. The method according to claim 6, wherein the control group is a group not treated with the candidate inhibitor of HVEM or the candidate inhibitor of HVEM ligand, and the candidate inhibitor of HSP, a group treated only with the candidate inhibitor of HVEM or the candidate inhibitor of HVEM ligand, or a group treated only with the candidate inhibitor of HSP.
